(19) United States
(12) Patent Application Publication
(10) Pub. No.: US 2010/0272710 A1
(43) Pub. Date: Oct. 28, 2010

(54) COMPOSITIONS AND METHODS FOR TREATING AND PREVENTING AGING-ASSOCIATED DISEASES

(76) Inventor: Abdelhadi Rebbaa, Guilderland, NY (US)

Correspondence Address: John Bianchi, 2804 Woodhaven Drive, Cinnaminson, NJ, 08077, US

(21) Appl. No.: 12/732,479

(22) Filed: Mar. 26, 2010

Related U.S. Application Data
(60) Provisional application No. 61/163,859, filed on Mar. 26, 2009.

(51) Int. Cl. A61K 39/395; A61K 31/7105; A61K 31/7088; A61K 31/195; A61K 31/445; A61K 31/047; A61K 31/215; A61K 38/00; A61K 38/22; A61P 3/10; A61P 35/00; A61P 25/28; A61P 3/04; A61P 9/10; A61P 19/10

(52) U.S. Cl. 424/130.1; 514/44 A; 514/44 R; 514/561; 514/315; 514/1.1; 514/9.7; 514/729; 514/529; 514/4.8; 514/16.4; 514/17.7; 514/15.4; 514/16.9; 514/19.3

(57) ABSTRACT

Provided herein are novel TXNIP-modulating compounds and methods of use thereof. The TXNIP-modulating compounds may be used to enhance cellular anti-stress defense and prevent stress-induced damage to DNA, proteins and lipids. Therefore, TXNIP-modulating compounds may be used to treat or prevent a wide variety of stress-induced diseases and thus aging.

### PRIORITY INFORMATION

This application claims priority to U.S. Provisional Application 61/163,859 under 35 U.S.C. §111(b).

### REFERENCES CITED

Alcendor R R, Gao S, Zhai P. “Sirt1 regulates aging and resistance to oxidative stress in the heart” Circ Res. 100:1512-1521 (2007).

Baur J A, Pearson K J, Price N L. “Resveratrol improves health and survival of mice on a high-calorie diet” Nature 444:337-342 (2006).

Bordone L, Guarente L. “Calorie restriction, SIRT1 and metabolism: understanding longevity” Nat. Rev. Mol. Cell Biol. 6:298-305 (2005).

Bordone L, Motta M C, Picard F. “Sirt1 regulates insulin secretion by repressing UCP2 in pancreatic beta cells” PLoS Biol. 4:e31 (2006).

Chen J. Saxena G, Mungrue I N et al. “Thioredoxin-interacting Protein: a Critical Link Between Glucose Toxicity and Beta Cell Apoptosis” Diabetes doi:10.2337/db07-0715 (Jan. 2, 2008).

Chondrogianni N, de C M Simoes D, Franceschi C, et al. “Cloning of Differentially Expressed Genes in Skin Fibroblasts from Centenarians” Biogerontology 5:401-409 (2004).

Chutkow W. A. et al. “Thioredoxin-interacting protein (TXNIP) is a Critical Regulator of Hepatic Glucose Production”. J Biol Chem. 283:2397-2406 (2008).

Farr S A, Banks W A, Uezu K. et al “DHEAS improves learning and memory in aged SAMP8 mice but not in diabetic mice” Life Sci. 75: 2775-85 (2004).

Fukui M, Kitagawa Y, Ose H et al. “Role of endogenous androgen against insulin resistance and athero-sclerosis in men with type 2 diabetes”. Curr. Diabetes. Rev. 3: 25-31 (2007).

Gescuk B D, Davis J C, Jr. “Novel therapeutic agents for systemic lupus erythematosus” Curr Opin Rheumatol 14:515-521 (2002).

Gutierrez G, Mendoza C, Zapata E, et al. [symbol] Dehydroepiandrosterone inhibits the TNF-alpha-induced inflammatory response in human umbilical vein endothelial cells” Atherosclerosis 190: 90-99 (2007).

Hasegawa K, Wakino S, Yoshioka K. “Sirt1 protects against oxidative stress-induced renal tubular cell apoptosis by the bidirectional regulation of catalase expression” Biochem Biophys Res Commun 372:51-56 (2008).

Hernandez-Morante J J, Perez-de-Heredia F, et al. “Role of DHEA-S on body fat distribution: gender- and depot-specific stimulation of adipose tissue lipolysis” Steroids 73(2):209-15 (2008).

Knight J. “The Biochemistry of Aging” Adv Clin Chem. 35:1-62 (200)

Lamberts S W, van den Beld A W, van der Lely A J. “The endocrinology of aging.” Science 278(5337):419-24 (1997).

Lan F, Cacicedo J M, Ruderman N, Ido Y. “SIRT1 modulation of the acetylation status, cytosolic localization, and activity of LKB1. Possible role in AMP-activated protein kinase activation” J. Biol. Chem. 283(41):27628-35 (2008).

Milne J C, Lambert P D, Schenk S. “Small molecule activators of SIRT1 as therapeutics for the treatment of type 2 diabetes” Nature 450:712-716 (2007).

Moynihan K A, Grimm A A, Plueger M M. “Increased dosage of mammalian Sir2 in pancreatic beta cells enhances glucose-stimulated insulin secretion in mice” Cell Metab 2:105-117 (2005).

Parikh H, Carlsson E, Chutkow W A “TXNIP Regulates Peripheral Glucose Metabolism in Humans” PLoS Med. 4:e158 (2007).

Patwari P, Higgins L J, Chutkow W A, et al. “The interaction of thioredoxin with TXNIP: Evidence for formation of a mixed disulfide by disulfide exchange. J Biol Chem. 281(31):21884-91 (2004).

Perrini S, Natalicchio A, Laviola L, et al. “Dehydroepiandrosterone stimulates glucose uptake in human and murine adipocytes by inducing GLUT1 and GLUT4 translocation to the plasma membrane” Diabetes 53: 41-52 (2004).

Qi W, Chen X, Gilbert R E, et al. “High glucose-induced thioredoxin-interacting protein in renal proximal tubule cells is independent of transforming growth factor-betal” Am J Pathol 171(3):744-54 (2007).

Schulze P. C. et al. (2004). “Hyperglycemia Promotes Oxidative Stress Through Inhibition of Thioredoxin Function by Thioredoxin-interacting Protein” J. Biol. Chem. 279 (29): 30369-74 (2004).

Smith J. “Human Sir2 and the ‘silencing’ of p53 activity” Trends Cell Biol. 12:404-406 (2002).

Trapp J, Jung M. “The role of NAD+ dependent histone deacety-lases (sirtuins) in ageing” Curr Drug Targets. 7:1553-1560 (2006).

Vijg J, Suh Y. Genetics of longevity and aging. Annu. Rev. Med. 56:193-212 (2005).

Wang F, Nguyen M, Qin F X, Tong Q. “SIRT2 deacetylates FOXO3a in response to oxidative stress and caloric restriction” Aging Cell 6:505-514 (2007).

Yoshida T., Kondo N., and Oka S. “Thioredoxin-binding Protein-2 (TBP-2): Its Potential Roles in the Aging Process” Biofactors 27:47-51 (2006).

Yoshioka J. “Targeted Deletion of Thioredoxin-interacting Protein Regulates Cardiac Dysfunction in Response to Pressure Overload” Circ. Res. 101:1328-1338 (2007)

### BACKGROUND

1. Technical Field

The present invention relates to the field of aging. In particular, the present invention relates to pharmaceutical compositions and methods for treating and preventing aging-associated diseases.

2. Summary

Provided herein are novel TXNIP-modulating compounds and methods of use thereof. The TXNIP-modulating compounds may be used to enhance cellular anti-stress defense and prevent stress-induced damage to DNA, proteins and lipids. Therefore, TXNIP-modulating compounds may be used to treat or prevent, or both, a wide variety of stress-induced diseases and disorders, for example, diseases or disorders related to aging including but not limited to cancer, diabetes, obesity, neurodegenerative diseases, cardiovascular disease, inflammation, as well as diseases or disorders that would benefit from increased oxidative damage. Also provided are compositions comprising a TXNIP-modulating compound in combination with another therapeutic agent. The object of the invention is for improving healthy aging and the extension of the organism's life span.

### DESCRIPTION OF THE FIGURES

FIG. 1.

Schematic describing the method used for the search of anti-stress genes. A cDNA library was made from drug resistant cancer cells and transfected in stress sensitive cells. After selection in the presence of stress conditions that inhibit proliferation, cells that continued to proliferate were isolated and cDNA inserts contained within these cells were sequenced.

FIG. 2.

Characterization of a clone that escaped stress toxicity.

FIG. 2A._Parental (wild type, WT) and cDNA-transfected and cells (clone 14, C14) were compared for resistance to H2O2.

FIG. 2B The terminal, middle and the C-terminal sections of the TXNIP gene were amplified by PCR from both the wild type cells (WT) or the clone C14. GADPH was used as a control.

FIG. 2C Western blot showing expression of the protein TXNIP between parental (WT) and the clone C14. beta actin was used as a control.

FIG. 2D A comparison of thioredoxin activity between parental (WT) and the clone C14.

FIG. 3.

Schematic representation highlighting the opposite effects of Sirt1 and TXNIP on oxidative stress and metabolism. Sirt1 has been shown to inhibit oxidative stress and improves glucose uptake as well as insulin secretion. In contrast, TXNIP was found to enhance cellular susceptibility to oxidative stress and inhibit glucose uptake and insulin secretion.

FIG. 4.

Effects of limited glucose availability, resveratrol and DHEA on expression of TXNIP and Sirt1 in cancer cells.

FIG. 4A.

SaOS2 cells were incubated in the presence of reduced glucose levels in the culture medium for 48 hours. Expression of TXNIP and Sirt1 were determined by Western blot using specific antibodies. Antibody to _-Actin was used as a loading control.

FIG. 4B.

Respective expression of TXNIP and Sirt1 in response to limited glucose availability, measured by quantitative real time PCR (Q-PCR). SaOS2 cells were incubated in the presence of the indicated amounts of glucose for 48 hours; QPCR was then performed to detect expression of TXNIP and Sirt1 using specific primers.

FIGS. 4C and 4D.

The RGC (4C) and SaOS2 (4D) cells were subjected to treatment with increasing concentrations of resveratrol (Resv.) for 48 hours followed by Western blot as described in Panel A.

FIG. 4E.

SaOS2 cells were treated with increasing amounts of DHEA and probed for expression of Sirt1 and TXNIP; beta-actin was used as a loading control.

FIG. 5.

Effects of limited glucose availability, resveratrol and DHEA on expression of TXNIP and Sirt1 in normal cells. Aortic smooth muscle cells (4A) and human embryonic stem cells (4B) were treated as described in FIG. 1 for cancer cells. Expression Sirt1 and TXNIP were analyzed by Western blot using specific antibodies. Antibody to GAPDH was used as loading control. (Glc: glucose; Resv: Resveratrol).

FIG. 6.

Tissue distribution of Sirt1 and TXNIP in mice and their regulation by resveratrol and DHEA. Mice CD1 strain were injected (i.p.) with 10 mg/Kg of either resveratrol or DHEA and after 2 days, the animals were sacrificed and organs harvested and processed by Western blot for the expression of Sirt1 and TXNIP. Staining with GAPDH was used as a loading control.

FIG. 7.

Putative mechanism(s) by which DHEA inhibits TXNIP.

FIG. 7A

SaOS2 (7A) were pre-incubated with Tamoxifen or CCP for one hour prior to addition of DHEA. After an additional incubation for 48 hours, proteins were extracted and probed by western blot for the expression of TXNIP and Sirt1.

FIG. 7B

The cells were treated with different concentrations of glucose with or without DHEA. After 48 hours in cultures, proteins were separated by electrophoresis and probed for TXNIP. Staining with beta-actin antibody was used as a loading control.

FIG. 7C.

Effect of DHEA and resveratrol on G6PD activity. Protein extract (100 _g) was incubated with DHEA or resveratrol at the indicated concentrations, after 30 min at 37° C., the activity of G6PD was measured as described in the methods section. Data represent average of four determinations +/−SE.

FIG. 8.

Effect of Resveratrol and DHEA on phosphorylation of AMPK. Panel A.

FIG. 8A

SaOS2 cells were treated with increasing concentrations of resveratrol for 48 hours, after what, proteins were extracted and probed either with anti-phospho-AMPK (Thr172), or with antibody to AMPK.

FIG. 8B.

RGC cells were incubated with 20 or 100 μM resveratrol or DHEA 100 μM for 48 hours, and then probed for phosphor-AMPK and AMPK as described above.

FIG. 9.

Regulation of TXNIP through the glycolytic pathway and its modulation by limited glucose availability (LGA), resveratrol and DHEA. Down regulation of glucose levels is known to increase the AMP/ATP ratio which in turn activates AMPK, leading to increased serine phosphorylation of ChREBP. Phosphorylated ChREBP is unable to translocate into the nucleus and form a functional complex with Mlx that is required for TXNIP expression. In addition, reduced glucose levels may result, the pentose pathway in the inhibition of the phosphatase PP2A. This will also result in the accumulation of phosphorylated ChREBP in the cytoplasm and further inhibition of TXNIP expression. DHEA acts on this glycolytic pathway mainly through the inhibition of G6PD activity. Resveratrol would act through the induction of Sirt1-mediated phosphorylation of AMPK, leading to enhanced phosphorylation ChREBP and inhibition of its nuclear translocation. This mechanism sheds light on TXNIP as a common downstream target for putative anti-aging interventions that affect metabolism.

### DETAILED DESCRIPTION OF THE INVENTION

It is well established that over time, the accumulation of damage to DNA (i.e. mutations, deletions, insertions, rearrangements), to proteins (i.e. oxidation, glycation, nitrosylation) and to lipids (i.e. peroxidation) represents the major cause of many chronic illnesses including but not limited to cancer, neurodegenerative diseases, heart diseases, diabetes, and osteoporosis. Since these damages are often caused by environmental (e.g., radiation, pollution, oxidation, chemicals) or endogenous stimuli (calorie intake, emotional stress, autoimmune response, e.g.), it is conceivable that protecting an organism's cellular ability to regulate oxidative stress and metabolism against stress-induced damage should prevent the onset of aging-associated diseases and delay the functional decline associated with the old age. However, to date, few genes capable of protecting normal cells against stress-induced damage have been discovered. One reason for this resides in the fact that potential sources for such genes are not yet known.

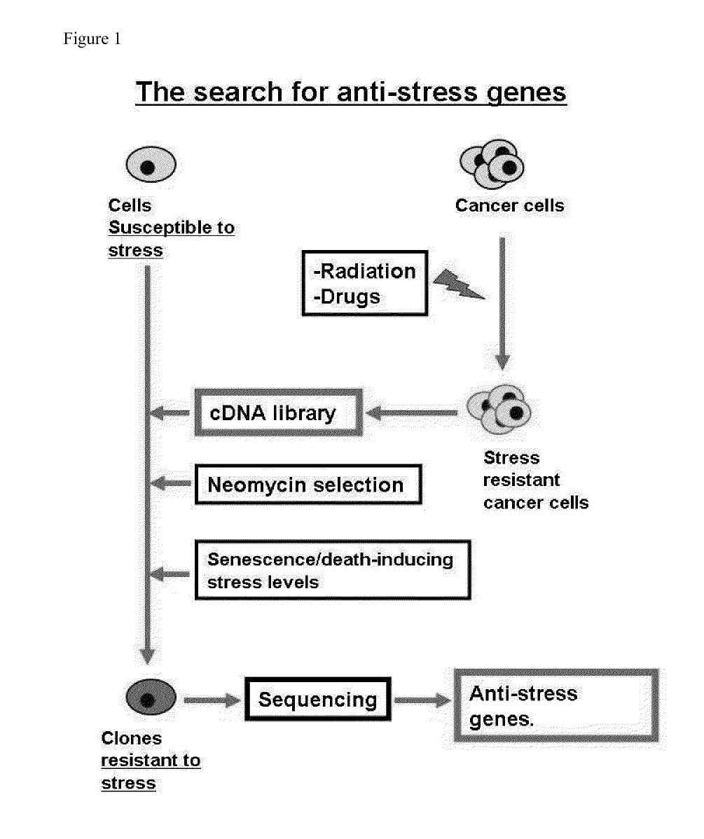

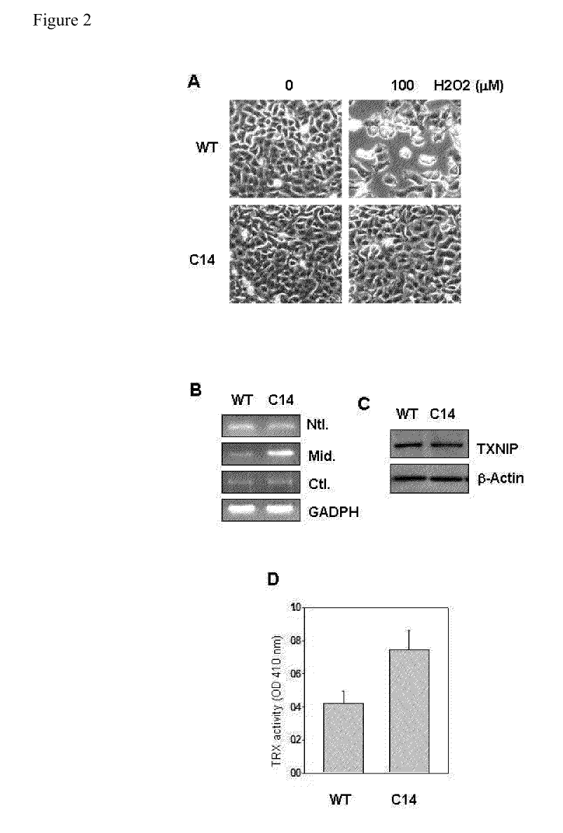

Previous work from our laboratory and others has demonstrated that cancer cells have the unique ability to adapt and become resistant to virtually any type of stress (including radiation and chemotherapy), suggesting that these cells may possess potent anti-stress defense. Based on this, we hypothesized that drug- or radiation-resistant cancer cells may represent a compelling source for the discovery of potent anti-stress genes. These genes, if introduced in normal cells, should prevent stress-induced alterations to DNA proteins and lipids and thus, suppress or at least delay the onset of stress-associated diseases and extend the organism's life span. To identify such genes, we have generated a cDNA library from drug-resistant cancer cells (FIG. 1) and used it to transfect stress-sensitive cells. The transfected cells were subjected to a stress that inhibits proliferation, and the clones that became resistant to stress were chosen. The cDNA inserts within these clones were sequenced. Initial screening of the library led to the discovery of candidate genes, one of them was a sequence coding for a dominant negative form of the thioredoxin interacting protein, TXNIP (FIG. 2). The clone #14 containing this sequence was found to be resistant to stress (FIG. 2A) and analysis of the sequence revealed that it corresponds to the middle region of the TXNIP gene (FIG. 2B). Although the protein level of TXNIP did not change (FIG. 2D), the activity of thioredoxin, a TXNIP target was enhanced (FIG. 2D). This indicates that the TXNIP sequence contained with the clone C14 acts as a dominant negative TXNIP.

A report by Patwari P, Higgins L J, Chutkow W A, et al. “The interaction of thioredoxin with TXNIP: Evidence for formation of a mixed disulfide by disulfide exchange. J Biol Chem. 281(31):21884-91 (2004) has demonstrated that TXNIP enhances cellular susceptibility to oxidative stress through direct interaction with the anti-stress enzyme, thioredoxin, and inhibition of its detoxifying functions. Over-expression of this gene was postulated by Chen J. Saxena G, Mungrue I N et al. “Thioredoxin-interacting Protein: a Critical Link Between Glucose Toxicity and Beta Cell Apoptosis” Diabetes doi:10.2337/db07-0715 (Jan. 2, 2008) to cause the accumulation of reactive oxygen species and apoptotic cell death. Hyperglycemic oxidative stress was demonstrated by Schulze P. C. et al. (2004). “Hyperglycemia Promotes Oxidative Stress Through Inhibition of Thioredoxin Function by Thioredoxin-interacting Protein” J. Biol. Chem. 279 (29): 30369-74 (2004). More recently, evidence was provided that TXNIP may also act as a mediator of cellular metabolism. For instance, this gene was found to mediate glucose-induced apoptotic death in pancreatic beta cells, suggesting a causative relationship between TXNIP and type 2 diabetes (Qi W, Chen X, Gilbert R E, et al. High glucose-induced thioredoxin-interacting protein in renal proximal tubule cells is independent of transforming growth factor-betal. Am J Pathol 171(3):744-54 (2007). Published papers by Yoshioka J. “Targeted Deletion of Thioredoxin-interacting Protein Regulates Cardiac Dysfunction in Response to Pressure Overload” Circ. Res. 101:1328-1338 (2007); Parikh H, Carlsson E, Chutkow W A “TXNIP Regulates Peripheral Glucose Metabolism in Humans” PLoS Med.4:e158 (2007); and Chutkow W. A. et al. “Thioredoxin-interacting protein (TXNIP) is a Critical Regulator of Hepatic Glucose Production”. J Biol Chem. 283:2397-2406 (2008) TXNIP deficiency improved glucose uptake and attenuated diabetic symptoms in animals. An inverse correlation between TXNIP expression and longevity is reported by Chondrogianni N, de C M Simoes D, Franceschi C, et al. “Cloning of Differentially Expressed Genes in Skin Fibroblasts from Centenarians” Biogerontology 5:401-409 92004); and Yoshida T., Kondo N., and Oka S. “Thioredoxin-binding Protein-2 (TBP-2): Its Potential Roles in the Aging Process” Biofactors 27:47-51 (2006). Accordingly, the dominant negative form of TXNIP may act to enhance life span.

Manipulation of metabolism and resistance to oxidative stress has been shown to promote longevity of small organisms (Vijg J, Suh Y. Genetics of longevity and aging. Annu. Rev. Med. 56:193-212 (2005), Knight J A. The biochemistry of aging. Adv Clin Chem. 2000; 35:1-62 (2000), and to some extent, these same mechanisms appear to act also in mammals despite considerable divergence during evolution. One of the most described genes that inhibit oxidative stress and improve glucose metabolism is the histone deacetylase Sirt1 (for Silent information regulator T1). Evidence has been provided recently that Sirt1 negatively regulates oxidative stress (Smith J. “Human Sir2 and the ‘silencing’ of p53 activity” Trends Cell Biol. 12:404-406 (2002); Hasegawa K, Wakino S, Yoshioka K. “Sirt1 protects against oxidative stress-induced renal tubular cell apoptosis by the bidirectional regulation of catalase expression” Biochem. Biophys. Res Commun. 372:51-56 (2008); Wang F, Nguyen M, Qin F X, Tong Q. “SIRT2 deacetylates FOXO3a in response to oxidative stress and caloric restriction” Aging Cell 6:505-514 (2007)) and protects cells against damage induced by H₂O₂, UV radiation, chemicals, and high caloric intake (Alcendor R R, Gao S, Zhai P. Sirt1 regulates aging and resistance to oxidative stress in the heart. Circ Res. 100:1512-1521 (2007), Baur J A, Pearson K J, Price N L. “Resveratrol improves health and survival of mice on a high-calorie diet” Nature 444:337-342 (2006)). In addition, this gene was found to regulate cellular metabolism through the stimulation of glucose uptake (Trapp J, Jung M. “The role of NAD+ dependent histone deacetylases (sirtuins) in ageing” Curr. Drug Targets 7:1553-1560 (2006), Milne J C, Lambert P D, Schenk S. “Small molecule activators of SIRT1 as therapeutics for the treatment of type 2 diabetes” Nature 450:712-716 (2007) and insulin secretion (Bordone L, Guarente L. “Calorie restriction, SIRT1 and metabolism: understanding longevity” Nat. Rev. Mol. Cell Biol. 6:298-305 (2005); Bordone L, Motta M C, Picard F. “Sirt1 regulates insulin secretion by repressing UCP2 in pancreatic beta cells” PLoS Biol. 4:e31 (2006), Moynihan K A, Grimm A A, Plueger M M. “Increased dosage of mammalian Sir2 in pancreatic beta cells enhances glucose-stimulated insulin secretion in mice” Cell Metab. 2:105-117 (2005)), making it a promising target of putative anti-aging interventions.

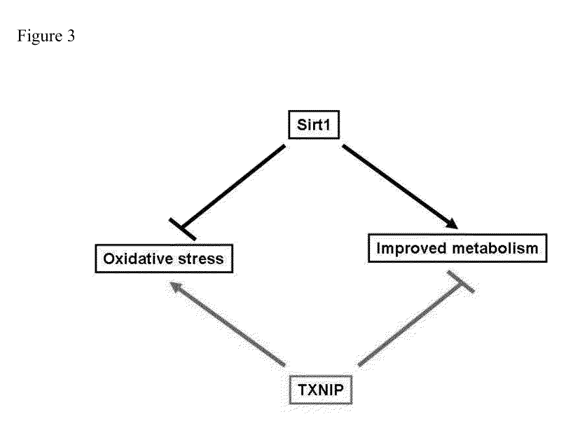

Also, taking into account this antagonistic relationship between Sirt1 and TXNIP regarding their effects on oxidative stress and metabolism (FIG. 3), it is important to determine if, and how putative anti-aging approaches would affect the function of these two genes, and whether a regulatory relationship might exist between them. To address these possibilities, we investigated the effects of limited glucose availability (used here to mimic the effects of calorie restriction), the Sirt1 activator (resveratrol), and the hormone dehydroepiandrosterone (DHEA), on expression of Sirt1 and TXNIP in different cellular systems, including cancer, stem cells and a mice model. A potential link between these two genes and the underlying mechanisms leading to their regulation were also investigated. Our results indicated that each of the treatment approaches tested exerted effects on the expression of Sirt1 and TXNIP, however to various degrees. Interestingly limited glucose availability was the only approach that consistently reduced TXNIP expression in all the systems studied. Resveratrol and DHEA exerted inhibitory effects on TXNIP in a tissue specific manner, in addition, they were found to act on separate branches of the glycolytic pathway mediated respectively by AMPK and Glucose 6 phosphate dehydrogenase respectively.

### DETAILED DESCRIPTION OF THE INVENTION

The results indicated that each of the treatment approaches tested exerted effects on the expression of Sirt1 and TXNIP, however, to various degrees. Limited glucose availability was the only approach that consistently reduced TXNIP expression in all the systems studied. Resveratrol and DHEA exerted inhibitory effects on TXNIP in a tissue specific manner, and further were found to act on separate branches of the glycolytic pathway mediated respectively by AMPK and Glucose 6 phosphate dehydrogenase, respectively.

### Examples

### Example 1

### Limited Glucose Availability, Resveratrol and DHEA Regulate the Expression of Sirt1 and TXNIP

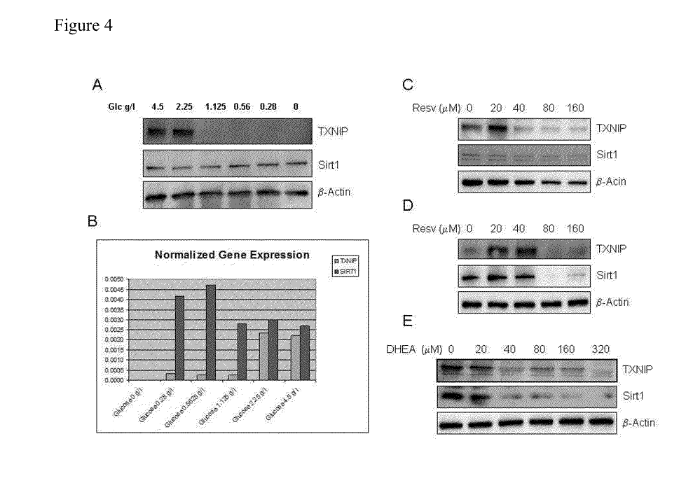

The effects of limited glucose availability (used here to mimic the effect of calorie restriction), resveratrol (an activator of Sirt1) and DHEA (used for hormone replacement therapy), on the expression of TXNIP and Sirt1 were determined by Western blot using specific antibodies to the corresponding proteins (FIG. 4). The data shows that cellular incubation with decreasing concentrations of glucose resulted in a dramatic loss of TXNIP expression (FIG. 4A). Similar results were observed by using other cancer cell lines (data not shown). Limited glucose availability was also found to cause a slight increase in the expression of Sirt1 (FIG. 4A), supporting the notion that this gene acts as a mediator of caloric restriction (12). PCR analysis (FIG. 4B) confirmed the previous findings and suggests that altered expression of TXNIP and Sirt1 in response to glucose deprivation occurred at the transcriptional level.

Curiously, resveratrol had a biphasic effect on the expression of TXNIP, with a stimulatory action at low concentrations and inhibition at higher ones (FIGS. 4C and 4D). Even more unexpected was the finding that resveratrol suppressed the expression of its own target, Sirt1, in both RGC cells (FIG. 4C) and SaOS2 cells (FIG. 4D), at the same concentrations that inhibit TXNIP. These data suggest that regardless of the concentration used, resveratrol may exert unwanted effects. At low concentrations, it induces the expression of TXNIP, a potential antagonist with regards to Sirt1 effect oxidative stress and metabolism, and at high concentrations, it inhibits expression of its own target. Based on this, the dosage at which resveratrol is administered should be carefully determined in order to achieving beneficial effects of this molecule.

Dehydroepiandrosterone, DHEA is a hormone thought to play a role in aging based on the observation that its levels decline dramatically with age to reach values of 30 to 20% at 70˜80 years (Lamberts S W, van den Beld A W, van der Lely A J. “The endocrinology of aging.” Science 278(5337):419-24 (1997)). Although some studies have suggested that replacement of this hormone at later age may have beneficial effects on illnesses such as atherosclerosis (Fukui M, Kitagawa Y, Ose H et al. “Role of endogenous androgen against insulin resistance and atherosclerosis in men with type 2 diabetes” . Curr. Diabetes. Rev.3: 25-31 (2007)), autoimmune diseases (Gescuk B D, Davis J C, Jr. “Novel therapeutic agents for systemic lupus erythematosus” Curr Opin. Rheumatol. 14:515-521 (2002)), obesity (Hernandez-Morante J J, Perez-de-Heredia F, et al. “Role of DHEA-S on body fat distribution: gender- and depot-specific stimulation of adipose tissue lipolysis” Steroids 73(2):209-15 (2008), and neurodegeneration (Farr S A, Banks W A, Uezu K. et al “DHEAS improves learning and memory in aged SAMP8 mice but not in diabetic mice” Life Sci. 75: 2775-85 (2004)), many studies failed to demonstrate any beneficial effect of this hormone on aging. At the molecular level, DHEA was shown to improve glucose uptake (Perrini S, Natalicchio A, Laviola L, et al. “Dehydroepiandrosterone stimulates glucose uptake in human and murine adipocytes by inducing GLUT1 and GLUT4 translocation to the plasma membrane” Diabetes 53: 41-52 (2004)) and reduce the formation of reactive oxygen species (Gutierrez G, Mendoza C, Zapata E, et al. “Dehydroepiandrosterone inhibits the TNF-alpha-induced inflammatory response in human umbilical vein endothelial cells” Atherosclerosis 190: 90-99 (2007)); however, the putative roles of Sirt1 or TXNIP in mediating these actions are not known. Here we show that DHEA acts as a dual regulator of these two genes (FIG. 4E). Exposure to increasing concentrations of this hormone resulted in decreased expression of both TXNIP and Sirt1. While the inhibition of TXNIP by this hormone is desirable, that of Sirt1 is not, suggesting that the anti-aging effects of DHEA, if any, would be hindered by its inhibitory effect on Sirt1 and related pathways.

Overall, the findings presented in FIG. 4 indicate that reduced calorie intake would rank the best among the three approaches tested in this system. In contrast, suppression of Sirt1 by both resveratrol and DHEA may represent a major limitation to their potential use as anti-aging therapeutics. Nevertheless, a proper understanding of how these two treatments inhibit Sirt1 will help in the development of approaches to avoid this “side effect” and improve their efficacy.

### Example 2

### Sirt1 and TXNIP are Expressed in Normal Cells

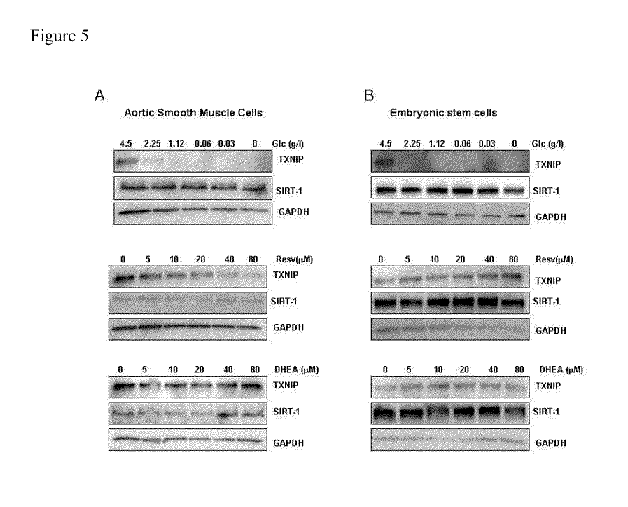

In order to obtain information on the physiological relevance of targeting Sirt1 and TXNIP in normal tissues, we investigated the effect of limited glucose availability, resveratrol and DHEA in differentiated aortic smooth muscle cells, and non-differentiated, human embryonic stem cells. As shown in FIG. 5A, limited glucose availability inhibited TXNIP expression in smooth muscle cells, without any significant effect on Sirt1. Similar effects were also observed with resveratrol which inhibited the expression of TXNIP in a dose dependent manner. DHEA was without effect on any of the two genes (FIG. 5A), raising the possibility that the glycolytic pathway targeted by this hormone may be altered in normal versus cancer cells. Of note, no significant changes in Sirt1 expression was observed in cells treated with any of the approaches tested, further confirming the difference in response to these treatment between normal and cancer cells.

To test whether cellular proliferative capacity may account for this difference, we measured expression of Sirt1 and TXNIP in embryonic stem cells known for rapid self renewal ability. The human embryonic stem cells, BG01V, were subjected to glucose restriction or treatment with either resveratrol or DHEA under similar conditions to those described in FIG. 4. As shown in FIG. 3B, both Sirt1 and TXNIP were found to be expressed in human embryonic stem cells; however, none of these genes was affected by resveratrol or DHEA. In contrast, limited glucose availability strongly inhibited TXNIP with no significant effect on Sirt1 expression, mirroring its action on cancer (FIG. 4) and smooth muscle cells (FIG. 5A). Taken together, these findings suggest that the pathways targeted by resveratrol and DHEA may be altered in cancer versus normal cells, and that the effects of these two treatment may be tissue specific. In contrast, the consistent inhibition of TXNIP by limited glucose availability in cancer cells as well as in differentiated smooth muscle cells and non-differentiated stem cells, is indicative of a potential role of this gene in mediating the anti-aging action of calorie restriction.

### Example 3

### Resveratrol and DHEA Affect Expression of Sirt1 and TXNIP in vivo

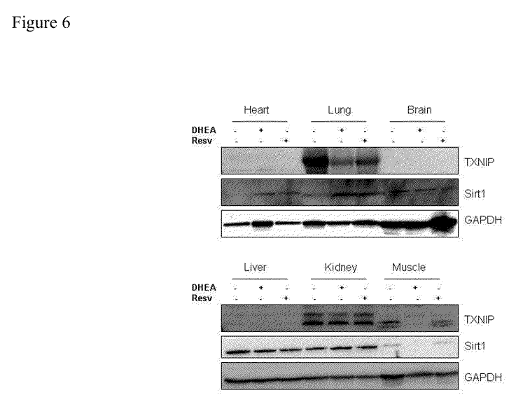

In order to determine whether the observed effects in vitro could be also valid in vivo, we have subjected the nude mice (CD1 strain, Charles River) to treatment with either resveratrol or DHEA (10 mg/Kg each) for 2 days. The animals were then sacrificed and major organs harvested to analyze expression of Sirt1 and TXNIP by Western blot. As shown in FIG. 6, the relative expression of these two genes was tissue specific. While Sirt1 is expressed in most organs tested; TXNIP was found to be expressed mainly in the lung, kidney and the muscle. Both resveratrol and DHEA inhibited the expression of TXNIP in the lung, however only DHEA had an inhibitory effect in the muscle. Sirt1 was not significantly affect in most tissues except in the muscle of animals treated with DHEA. Taken together, these findings suggest that resveratrol and DHEA are capable of regulating the expression of TXNIP and Sirt1, however in a tissue-specific manner.

### Example 4

### Comparative Effects of DHEA and Resveratrol on Activation of the Glucose 6 Phosphate Dehydrogenase

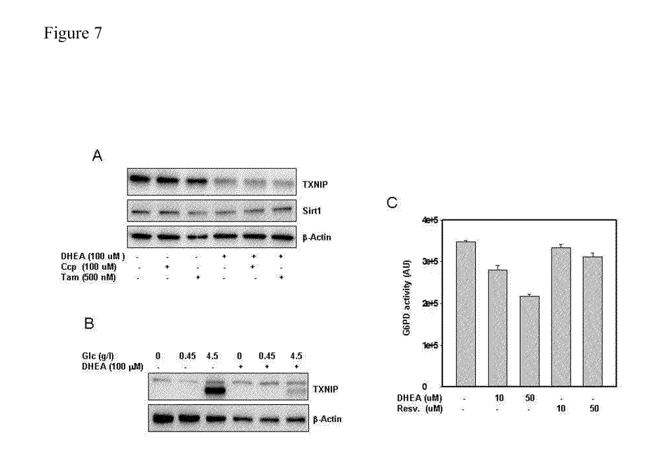

Independently of the putative mechanisms described above, we have observed that cellular pre-treatment with DHEA almost completely blocked the effects of glucose on TXNIP expression (FIG. 7B), suggesting that this hormone may act, at least in part, by interfering with the glycolytic pathways that regulate TXNIP expression. Since DHEA has also been shown to inhibit the glucose 6 phosphate dehydrogenase, G6PD (20), we investigated whether this possibility may account for the observed effects of this hormone on TXNIP. The results presented in FIG. 7C show that the activity of G6PD was indeed strongly inhibited and in a dose dependent manner by DHEA. Of note, since this in vitro assay is based on the production of NADPH (half of which is produced by G6PD and the rest by other enzymes such as malic acid dehydrogenase and isocitrate dehydrogenase (21)), the data (FIG. 7C) suggest that the inhibitory effect of DHEA on G6PD could be much higher than the 50% observed. Resveratrol has no effect on G6PD (FIG. 7C) suggesting that it may act through a different pathway to regulate TXNIP.

### Example 5

### Both DHEA and Resveratrol Regulate the AMP-Activated Protein Kinase

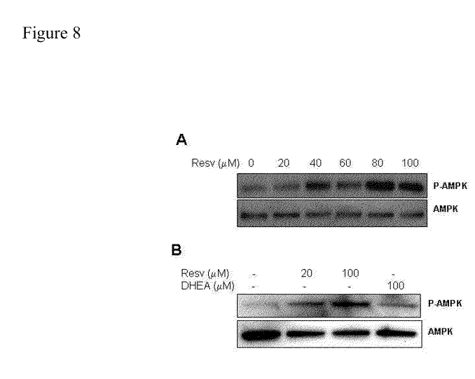

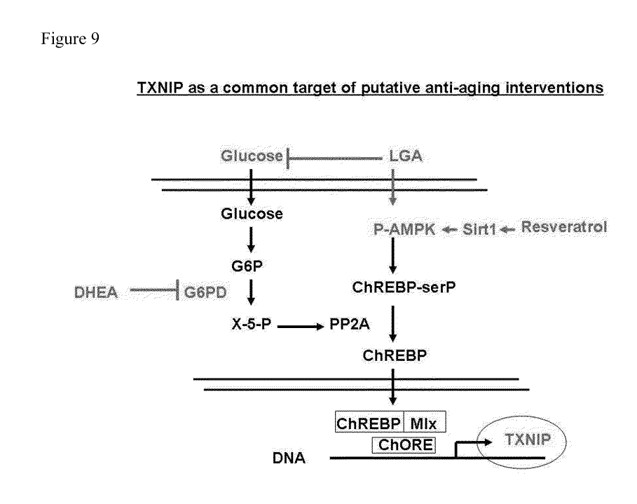

Recent investigations of the mechanisms by which resveratrol improves metabolism revealed that it induces phosphorylation of the AMP-mediated protein kinase (AMPK) (Dasgupta B, Milbrandt J. “Resveratrol stimulates AMP kinase activity in neurons” PNAS 104: 7217-7222 (2007)), an enzyme that phosphorylates and inactivates the carbohydrate response element binding protein ChREB. However, the relevance of this action to the regulation of TXNIP by resveratrol and the possible implication of DHEA in regulating this pathway are not yet investigated. The results presented in FIG. 6A show that resveratrol readily activates this enzyme (p-AMPK) in the retinal glial cells (RGC) without affecting expression of the corresponding gene (AMPK). Resveratrol also induced AMPK phosphorylation in the osteosarcoma cells SaOS2 (FIG. 8B), however DHEA was without effect. These findings, in addition to those presented in FIG. 7D), suggest that, although both molecules inhibit TXNIP, DHEA and resveratrol act through separate branches of the glycolytic pathway mediated by G6PD and AMPK respectively. Sirt1 is able to activate AMPK (Lan F, Cacicedo J M, Ruderman N, Ido Y. “SIRT1 modulation of the acetylation status, cytosolic localization, and activity of LKB1. Possible role in AMP-activated protein kinase activation” J. Biol. Chem. 283(41):27628-35 (2008)), our data suggest that TXNIP may be regulated by Sirt1 through this pathway (FIG. 9). Further confirmation of this link will help establish TXNIP inhibition as an important determinant for evaluating the efficacy of putative anti-aging interventions. The finding that limited glucose availability is the only approach that consistently inhibited expression of TXNIP in all systems used in this study, is in agreement with this concept and suggests that, regardless of Sirt1 levels, decreased expression of TXNIP may have beneficial effects for delaying the aging process.

Overall, these findings provide evidence that all known anti-aging interventions tested here inhibit TXNIP therefore this gene represents a compelling target for anti-aging therapy.

It is preferred to administer a pharmaceutical composition comprising a pharmaceutically acceptable excipient, diluent, or carrier and a therapeutically effective amount of an: inhibitor of the expression and/or the function of the thioredoxin interacting protein (TXNIP/VDUP-2), or a salt thereof. The inhibitor may be a chemical molecule, or derived from siRNA oligonucleotides, Dominant negative peptides, SC-FV antibodies, hormones such as dehydroepiendroetene (DHEA), S-allylcysteine, Tempol, and polyphenols such as Resveratrol.

A therapeutically effective inhibitor of the expression and/or the function of the thioredoxin interacting protein (TXNIP/VDUP-2 provides diminished cellular susceptibility to oxidative stress and improves glucose uptake and insulin secretion.

A composition of the present invention may be administered in any desired and effective manner: as compositions for oral ingestion, or for parenteral or other administration in any appropriate manner such as intraperitoneal, subcutaneous, intratumoral, topical, intradermal, inhalation, intrapulmonary, rectal, vaginal, sublingual, intramuscular, intravenous, intra-arterial, intrathecal, or intralymphatic.

To protect against gastric or other secretion, the composition may be encapsulated or otherwise protected.

Further, the composition may be administered via implantation of a stent, or via direct injection into a tissue or organ.

Transfection and electroporation are also suitable routes of administration for compositions containing a nucleic acid.

It is preferred that regardless of the route of administration selected, the composition be formulated into pharmaceutically-acceptable dosage forms by conventional methods.

The dosage forms and methods are known to those of ordinary skill in the art (e.g., see: Remington's Pharmaceutical Sciences, Mack Publishing Co., Easton, Pa.). Pharmaceutical carriers are well known in the art (e.g., see: Remington's Pharmaceutical Sciences cited above and The National Formulary, American Pharmaceutical Association, Washington, D.C.) and include sugars (e.g., lactose, sucrose, mannitol, and sorbitol), starches, cellulose preparations, calcium phosphates (e.g., calcium hydrogenphosphate, dicalcium phosphate, and tricalcium phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and triglycerides), biodegradable polymers (e.g., polylactide-polyglycolide, poly[orthoesters], and poly[anhydrides]), elastomeric matrices, liposomes, nanoparticles, microspheres, oils (e.g., corn, germ, olive, castor, sesame, cottonseed, and groundnut), cocoa butter, waxes, paraffins, silicones, talc, salicylates, and the like.

It is preferred that carriers used included in the composition of the present invention be compatible with the other ingredients of the composition.

Carriers suitable for a selected dosage form and intended route of administration are well known in the art, and acceptable carriers for a chosen composition, dosage form and method of administration can be determined using ordinary skill in the art.

The composition of the present invention may, optionally, contain one or more additional agents commonly used in pharmaceutical compositions. These agents are well known in the art and include but are not limited to fillers or extenders, such as starches, lactose, sucrose, glucose, mannitol, silicic acid or the like; binders, such as carboxymethylcellulose, alginates, gelatin, polyvinyl pyrrolidone, hydroxypropylmethyl cellulose, sucrose, acacia or the like; humectants, such as glycerol or the like; disintegrating agents, such as agar-agar, calcium carbonate, potato or tapioca starch, alginic acid, certain silicates, sodium starch glycolate, cross-linked sodium carboxymethyl cellulose, sodium carbonate or the like; solution retarding agents, such as paraffin or the like; absorption accelerators, such as quaternary ammonium compounds or the like; wetting agents, such as acetyl alcohol, glycerol monostearate or the like; absorbents, such as kaolin, bentonite clay or the like; lubricants, such as talc, calcium stearate, magnesium stearate, solid polyethylene glycols, sodium lauryl sulfate or the like; suspending agents, such as ethoxylated isostearyl alcohols, polyoxyethylene sorbitol and sorbitan esters, microcrystalline cellulose, aluminum metahydroxide, bentonite, agar-agar, tragacanth or the like; buffering agents; excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, polyamide powder or the like; inert diluents, such as water, other solvents or the like; preservatives; surface-active agents; dispersing agents; control-release or absorption-delaying agents, such as hydroxypropylmethyl cellulose, other polymer matrices, biodegradable polymers, liposomes, microspheres, aluminum monostearate, gelatin, waxes or the like; opacifying agents; adjuvants; emulsifying and suspending agents; solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols, fatty acid esters of sorbitan or the like; propellants, such as chlorofluorohydrocarbons or the like and volatile unsubstituted hydrocarbons, such as butane, propane or the like; antioxidants; agents which render the formulation isotonic with the blood of the intended recipient, such as sugars, sodium chloride or the like; thickening agents; coating materials, such as lecithin or the like; and sweetening, flavoring, coloring, perfuming and preservative agents. Each such ingredient or material should be compatible with the other ingredients of the formulation. Agents suitable for a selected dosage form and intended route of administration are well known in the art, and acceptable ingredients and materials, dosage form and method of administration may be readily determined by those of ordinary skill in the art.

A composition in accordance with the present invention that is suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations can be prepared by methods well known in the art.

What is claimed is:

1. A composition comprising a pharmaceutically acceptable excipient, diluent, or carrier and a therapeutically effective amount of an: inhibitor of the expression or the function of the thioredoxin interacting protein (TXNIP/VDUP-2), or a salt thereof, derived from the group consisting of small molecules, siRNA oligonucleotides, Dominant negative peptides, SC-FV antibodies, hormones, S-allylcysteine, 4-hydroxy-2,2,6,6-tetramethylpiperidine-N-oxyl, and polyphenols;

2. The composition of claim 1 wherein the form is selected from the group consisting of capsule, sachet, pill, thin film, tablet, powder, granules, solution, suspension, liquid, emulsion, elixir, syrup, electuary, paste, tincture, pastille, injection, liposome, stent, implant, seed, microparticle, nanoparticle, patch, film, gel, cream, ointment, aerosol, spray, enema, douche, suppository, and pessary;

3. The composition of claim 1 wherein the hormone is dehydroepiandrosterone;

4. The composition of claim 1 wherein the hormone is an ester of dehydroepiandrosterone;

5. The composition of claim 1 wherein the hormone is a halogenated dehydroepiandrosterone;

6. The composition of claim 1 wherein the polyphenol is resveratrol;

7. The composition of claim 1 wherein the polyphenol is a resveratrol ester;

8. A method for preventing stress-induced cellular damage in a mammal; by administering to a mammal a composition of claim 1;

9. The method of claim 8 wherein the stress-induced cellular damage is to DNA, RNA, proteins, or lipids;

10. The method of claim 8 wherein the stress-induced cellular damage is selected from the group consisting of cancer, diabetes, metabolic syndrome, hepatic disease, in-born errors of metabolism, neurodegeneration, neuropathy, cardiovascular disease, myopathy, dystrophy, osteoporosis, immune disorder, renal disease, inflammatory disease, polyglutamine disease, respiratory disease, ischemia, coagulopathy, cachexia, and obesity;

11. A method for treating stress-induced cellular damage in a mammal needing such treatment by administering to the mammal needing such treatment a composition a composition of claim 1;

12. The method of claim 11 wherein the stress-induced cellular damage is to DNA, RNA, proteins, or lipids;

13. The method of claim 11 wherein the stress-induced cellular damage is selected from the group consisting of cancer, diabetes, metabolic syndrome, hepatic disease, in-born errors of metabolism, neurodegeneration, neuropathy, cardiovascular disease, myopathy, dystrophy, osteoporosis, immune disorder, renal disease, inflammatory disease, polyglutamine disease, respiratory disease, ischemia, coagulopathy, cachexia, and obesity;

14. A method for prolonging the lifespan of a mammal comprising administering to a mammal a composition of claim 1.
